(19) United States
(12) Patent Application Publication
(10) Pub. No.: US 2019/0348159 A1
(43) Pub. Date: Nov. 14, 2019

(54) ELECTRONIC HEALTH RECORD INTEROPERABILITY TOOL

(71) Applicant: Richard R. Selvaggi, Commerce, TX (US)

(72) Inventor: Richard R. Selvaggi, Commerce, TX (US)

(21) Appl. No.: 16/406,907

(22) Filed: May 8, 2019

Related U.S. Application Data
(60) Provisional application No. 62/668,449, filed on May 8, 2018.

(51) Int. Cl. G16H 10/60; G06F 16/16

(52) U.S. Cl. CPC G16H 10/60; G06F 16/164

(57) ABSTRACT

The disclosed principles provide for a new and unique electronic health record interoperability tool used to exchange patient data between disparate Electronic Health Record (EHR) systems. The disclosed tool provides systems and methods for breaking down EHR's into elementary data fields that can then be saved as individual data files, each file containing references to an originating EHR, the type of data stored in the file, and the field data itself. Existing EHR systems can be updated to both export and import medical records using the format of the disclosed tool to allow interoperability between differently configured EHR systems. Such data files can also be exported and stored on portable storage devices which can then be carried by the patient. The portable storage device can also be used as a smart card to quickly access, when possible, large data files stored in centralized or cloud-based storage.

### RELATED APPLICATION

The present disclosure claims priority to and is a non-provisional conversion of U.S. Provisional Patent Application No. 62/668,449, filed May 8, 2018, which is incorporated herein by reference in its entirety for all purposes.

### TECHNICAL FIELD

The present disclosure relates to electronic health records, and in particular to systems and methods for providing interoperability between differently structured or formatted electronic health records.

### BACKGROUND

Current medical practices keep records of patient data by recording information about patients and corresponding medical events in a Standard Medical Record (SMR). An SMR may be paper-based, or it could be stored electronically locally, at a central location, or also in the cloud. Formats of SMR's can vary, although the kinds of medical information contained within different SMR's tends to be of the same type. For example, there can be basic information to identify each patient, as well as records indicating the patient's blood pressure and other test results.

The vast majority of SMR's are migrating from paper-based records to Electronic Health Records (EHR's). Patient information in every EHR is organized into the same categories as in a paper-based SMR. The electronic data configuration of similar standard medical record information is different for each EHR system format, making sharing of electronic information difficult and impractical among all the various EHR formats available in the marketplace. Differences in structures between paper-based SMRs do not result in a problem when sharing medical records since this requires human interaction to interpret the contents of such SMRs. However, differences in the structural formats of EHRs pose a problem for the sharing of such EHRs since translation software is needed to convert the contents of one format type of EHR into an EHR formatted for a different system.

In order to increase efficiency and comply with new requirements to convert to EHR's, the vast majority of medical practices are entering all patient data electronically into their own unique EHR systems. These EHR systems are being developed by various companies who design their products to meet the individual needs of various medical organizations. The EHR's work well within the organizations who use the same EHR software systems, but cannot be readily shared with EHR's in systems independently developed by other companies. This form of incompatibility results in not being able to share electronic medical records between different medical organizations that are used by the patient when those organizations use different EHR systems. For example, a patient may be travelling when in need of medical attention, and while the patient's records may be accessible remotely at the travel site, the format of the medical data will likely be incompatible if different EHR systems are involved.

With the increasing development and use of EHR systems, there is need in the art for a way of sharing medical records to be able to efficiently and accurately transfer a patient's medical information electronically. However, as of the filing of this disclosure, there are no universal EHR interoperability tools available, nor are there any known future tools proposed that will provide a universal workable solution. A key problem is that there are literally hundreds of EHR products available with different technology architectures, file formats, service models, and capabilities. Consequently, the electronic data storage configuration of similar standard medical record information is generally different for each EHR system, making sharing of electronic information difficult and impractical.

One way of solving this problem is to create custom software to convert EHR's from one format directly to a format used by a different system. While this type of “transcoding” could be a workable solution for interoperability, it would only be useful between two specific systems for which the transcoding software has been created. In addition, the high cost of developing data interchange transcoding software that exchanges medical records directly between disparate EHR systems results in a lack of incentive to develop interoperability. Additionally, there is no standard for storing electronic medical records on a portable device in a way that enables sharing medical records between disparate EHR systems.

A preferred solution for interoperability between disparate EHR systems would be to devise a universal standard for an intermediate EHR file format, thus allowing software developers to create data interchange software to import and export EHR data using a standardized intermediate data interchange format. Once such interchange software is created for a particular EHR system, it would allow sharing of patient records with any other EHR system which can import and export patient records in the standardized data interchange format. One current effort for creating an intermediate EHR is to translate a complete EHR into a standardized format, such as the draft standard called Fast Healthcare Interoperability Resources (FHIR), proposed by Health Level Seven International. Unfortunately, however, the resulting intermediate EHR file structure is complex and difficult to implement. Modifying software for existing EHR systems to export and import these complex files is a considerable effort and cost. Thus, smaller EHR software companies or small healthcare provider offices with custom software would find the development cost and effort prohibitive.

Accordingly, what is needed is an EHR interoperability tool that allows electronic data to be shared between disparate EHR systems to provide an all-inclusive “super medical record” on an emergent basis. The most important time to allow sharing of electronic data occurs during emergencies, when time is of the essence and patients are least likely have the capacity to provide key health information or codes for access to centralized data storage. To be effective, such an interoperability tool and data exchange must allow for emergent and routine exchange of electronic health information. The disclosed principles establish simplified standard data interchange systems and methods that reduce the cost and effort required to enable EHR data sharing between disparate EHR systems.

### SUMMARY

To overcome the deficiencies of the prior art, the disclosed principles can be used in an EHR interoperability tool (herein referred to as “SCUTWORK”) that provides interoperability between disparate EHR systems. SCUTWORK provides a technique for breaking down EHR's into elementary data blocks that can then be easily imported into differently structured EHR systems. For example, the disclosed unique interoperability tool labels elemental data blocks of the standard electronic health record. These labels contain accountability information about the patient and the provider authoring the record along with identifying standard medical record information (e.g., provider, institution, date, patient, and elemental medical record data identifier). These labeled elemental data blocks can be cut from the structured EHR to create unstructured data stored on a portable memory device. SCUTWORK EHR data exported and stored on portable storage devices and carried by the patient enables sharing of medical records between non-connected and dissimilar EHR systems. Smart card features on the portable memory device allow for connectivity and ease of access to central or cloud record repositories for large data items such MRIs, CATs, angiograms, etc.

In order to create an efficient and standardized way of sharing patient data, an EHR is broken down into basic sets of data. To break the EHR into basic data sets, the standard medical record is divided into elemental parts, which are the various categories of information that would typically be found in an EHR. For example, a typical EHR would have Patient Demographics, which is the personal identifying information for the patient whose EHR is it, Practice Demographics, which is the identifying information of the place and provider that provided a medical procedure, etc. for the patient, and Procedure information, which is the specific information about the recorded procedure, such as date and time, medical/insurance code(s), and any related report, findings, films, etc. Of course, other categories of the information that may be found in a patient's health record could also be considered an elemental part of an EHR as used in accordance with the disclosed principles. The electronic data representing the individual data within each elemental part of the medical record becomes one basic data set. Each basic data set is extracted, labeled with a unique identifier (defined in detail below), and saved as a unique basic data file. These unique files can be imported and reassembled into an EHR in a different system.

The intermediate step provided by the disclosed principles of breaking down an EHR into unique basic data files for reassembling into another EHR solves the problem of incompatibility between disparate EHR systems. An important part of systems and methods in accordance with the disclosed principles is the creation of a unique identifier, referred to herein as an Accountability Identifier (A-I). The A-I is used to tie data exported from an EHR to a specific individual/provider and medical event. The A-I is composed of elements that uniquely identify, for example, the patient, the provider, time and date of service, location of service, type of service, and other relevant pieces of information. In this respect, the medical record(s) of the patient is/are used by the SCUTWORK tool to label the electronic data/files for storage as unstructured data. This A-I is attached as a header to each basic data set extracted from an EHR and saved as a unique basic data file. Since the unique data files are created as unstructured data, the A-I helps ensure that each individual exported data file can be properly restored to a complete original or equivalent EHR.

In addition to the A-I, another identifier is used to mark the type of data contained within each exported unique data file. A Field Identification Code (FIC) is included in each exported data field file, between the A-I header and the field data, to properly identify the source field of the EHR data contained within each exported data file. The FIC contained within the file ensures that the contents of each individual exported data file can be imported and inserted into the correct field when reconstructing an EHR from the previously exported data files. Accordingly, the A-I serves to identify each unique, unstructured data file, and the FIC serves to organize the raw data within the data file into proper fields based on the field in the EHR being broken down where the data was taken.

By creating the unique data files in this manner, the data files are simply unstructured data that does not contain a specific format or other structure since that would limit the ability of another system to read/interpret the data files based on its own formatting structure. Instead, by creating unstructured data files, existing EHR systems can be updated to both export and import medical records using the SCUTWORK format by simply a software update. Such an update would allow the updated EHR system to interpret the A-I and FIC in each unique, unstructured data file, and then properly rebuild the EHR using the raw data within each file. Once an EHR system is updated with SCUTWORK capabilities, there is no need to create conversion software to enable importing or exporting EHR records between two paired but incompatible EHR systems.

Current EHRs and other such medical IT-based systems use electronic files created from computer science concepts (i.e., a data structure providing data organization, management and storage format of a particular system) to label the medical record. As will become clear from the embodiments discussed below, SCUTWORK uses medical accountability concepts to label the electronic files and the data therein, providing a simple and inexpensive method for allowing interoperability among disparate medical record systems. Using the medical record to label the electronic files allows for the most beneficial SCUTWORK functions, including but not necessarily limited to, a “super medical record” that starts at birth and ends at death; immediate availability of the medical record for life-threatening emergencies; and the ability of providers to select specific types of records such as immunizations or a patient's white blood cell count over time. The ability to verify specific types of records also helps to avoid unnecessary duplication of services, thus aiding in the reduction of more than 25 billion dollars of medical waste a year by having access to the entire medical record when it is needed by the medical service provider.

SCUTWORK also operates by transforming the EHR from 100% structured data to 100% unstructured data and back again into 100% structured data. More specifically, as a first healthcare provider creates or updates an EHR of a patient, that data is structured data within that healthcare provider's system. When the EHR information is stored on behalf of the patient, the disclosed principles via the SCUTWORK tool allows storing of the patient's medical records as unstructured data. When any part or all of that medical record of the patient is accessed by a second healthcare provider, the SCUTWORK tool then converts the stored unstructured data back into structured data that is compatible with the system/format of the second healthcare provider. There are no known EHRs that use unstructured data and this is one of the reasons that there is no interoperability between different EHRs. This distinguishing feature of SCUTWORK is not limited to only the health field; any professional field that uses uniformly standardized records can benefit from the principles provided by SCUTWORK.

Numerous embodiments and advantages associated with each such embodiment are discussed in further detail below.

### BRIEF DESCRIPTION OF THE DRAWINGS

The detailed description that follows, by way of non-limiting examples of embodiments, makes reference to the noted drawings in which reference numerals represent the same parts throughout the several views of the drawings, and in which:

FIG. 1 illustrates a diagram of a configuration of an Accountability Identifier (A-I) in accordance with the disclosed principles as a file header to uniquely label each basic data set extracted from an EHR, and exported to an external file system;

FIG. 2 illustrates a diagram of individual data fields exported from an EHR in accordance with the disclosed principles to a first EHR system, where each individual data field contains an A-I header, a FIC indicating the source data field, and the actual data itself; and

FIG. 3 illustrates a diagram showing how a data field previously exported to a file from an EHR can be imported into a second EHR system, where fields identified as belonging to a specific EHR, as labeled by an A-I header, are further identified by an FIC that indicates the type of data contained within the file, and where the combination of the A-I with the FIC allows a differently structured EHR system to correctly reconstruct a patient's EHR from the individual data files.

The above-referenced figures are provided herein for the purpose of illustration and description only, and are not intended to define the limits of the disclosed principles.

### DETAILED DESCRIPTION

In view of the foregoing, through one or more various aspects, embodiments and/or specific features, the present disclosure is intended to bring out one or more of the advantages that will be evident from the description. The present disclosure makes reference to one or more specific embodiments by way of illustration and example. It is understood, therefore, that the terminology, examples, drawings and embodiments are illustrative and are not intended to limit the scope of the disclosure.

The scope of the present disclosure includes any feature or combination of features disclosed herein (either explicitly or implicitly), or any generalization thereof, whether or not it mitigates any or all of the problems addressed herein. Accordingly, new claims may be formulated during prosecution of this application (or an application claiming priority thereto) to any such combination of features. In particular, with reference to the appended claims, features from dependent claims may be combined with those of the independent claims and features from respective independent claims may be combined in any appropriate manner and not merely in the specific combinations enumerated in the appended claims.

The disclosed principles provide systems and methods for extracting individual fields from an Electronic Health Record (EHR), and storing each of the individual fields as a separate unique file external to, and using a format independent of, the EHR system. Each separate data file is marked with an Accountability Identifier (A-I) code to tie each EHR event (e.g., medical procedure, visit, prescription, examination, evaluation, or other information related to a patient's medical history) to a particular individual patient. As such, each EHR as used herein pertains to the record of a specific medical event of a particular patient, and therefore a patient will typically have many different EHRs throughout their medical history, the collection of which creates the entire medical history of a patient. In addition, each of the created data files is also marked with a Field Identifier Code (FIC) to indicate the field where the extracted EHR data originated. The combination of the A-I codes and FICs with the data in the external file allows an EHR to be accurately reconstructed from a set of files previously exported from an EHR.

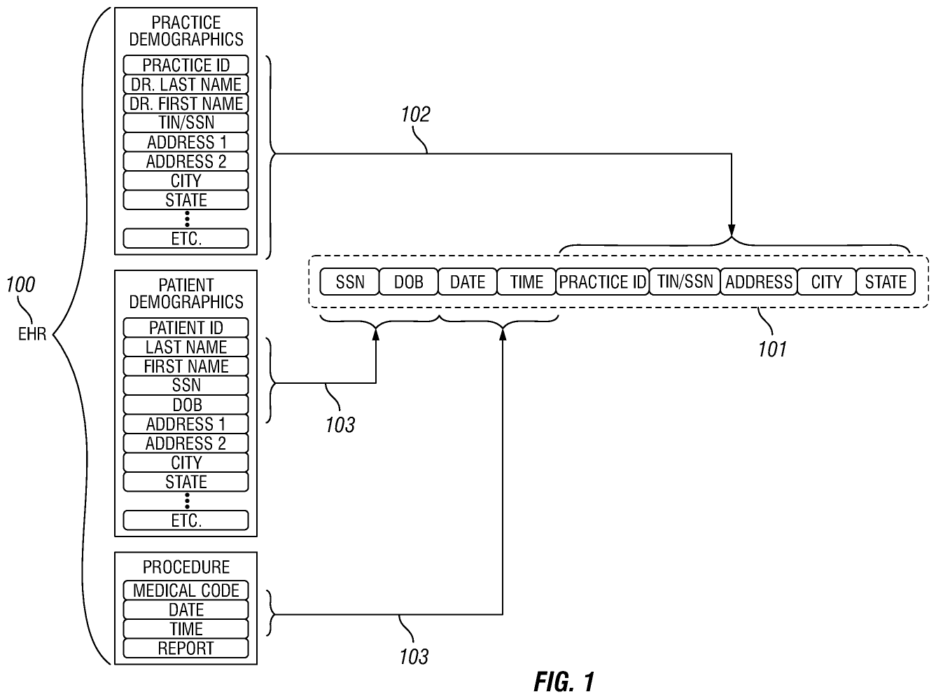

Looking more specifically at these components, in order to tie a set of exported EHR field files to a specific medical event, an A-I code is created from key fields of data within an EHR. FIG. 1 illustrates a diagram of a configuration of an A-I in accordance with the disclosed principles as a file header to uniquely and individually label each collection of data extracted from an EHR, and exported to an external file system. In particular, FIG. 1 shows an example of one possible implementation of an A-I 101 from a typical EHR 100. An EHR contains, in this exemplary embodiment, Medical Practice Demographics 102, Patient Demographics 103 and Procedure information 104. Key fields are extracted from the EHR from each of the types of information, and combined to create an identification code used as a label to tie extracted data fields to a specific EHR. The A-I code 101 can be used as a fixed length header at the start of each extracted data set to allow identification of all data sets/files related to a specific EHR.

The A-I information is unique for each label, but is the same for each label pertaining to particular EHR. Using the unique A-I results in a unique A-I label being generated by a software program for every elemental data set for that particular EHR. Each A-I field may be a fixed length, so the resulting A-I code would also be of a fixed length which would make it easier to parse. A careful design of the field labeling may be done to include all potential data type fields so that no data is lost when being exchanged between disparate EHR systems. Each field could be separated by a unique character, such as an exclamation point, which would not appear within normal data (e.g., not a period), followed by a termination character to indicate the end of the A-I string, such as a tilde (˜).

The A-I code would be a header at the beginning of a data file, which is used to identify a record associated with a specific individual, followed by data that is uniquely marked (by another header) to identify the type of data within the file. In advantageous embodiments, the information in the A-I should be comprehensive enough to uniquely tie the medical service, procedure, report, etc. to each individual patient without being so narrowly tied to a single event that it would exclude searching for common procedures, such as searching for physical examinations or immunization records over time. Similarly, to allow for searching for specific medical events over time, information that may change over time, such as an address, practice location, physician, etc. should not be included within the A-I. The FIC allows searches for medical information of a patient, while the A-I allows searches of accountability.

Part of the A-I code/header may include a string of concatenated characters (numbers & letters) which uniquely identify an individual, using the minimum amount of data to identify the individual. For example, the minimum data for a unique A-I may include unique information pertaining to the patient and to the particular EHR being deconstructed. Moreover, as discussed above, fixed lengths may be assigned for each bit of information comprising the A-I, and arranged in a predetermined order to identify what each separate part of the A-I code indicates. Examples of possible portions of an A-I code are set forth in Table 1 below.

TABLE 1

| A-I CODE PART | UNIQUE INFORMATION |
|---|---|
| PANA****** | Patient Name (Max Field Size) |
| SS#### | Patient Social Security Number (Partial) |
| DB####-##-## | Patient Date of Birth in YYYY-MM-DD Format |
| PRNA****** | Name of Service Provider (Max Field Size) |
| PRLO****** | Location of Medical Event or Procedure |
| ME####-##-## | Date of Medical Event in YYYY-MM-DD Format |

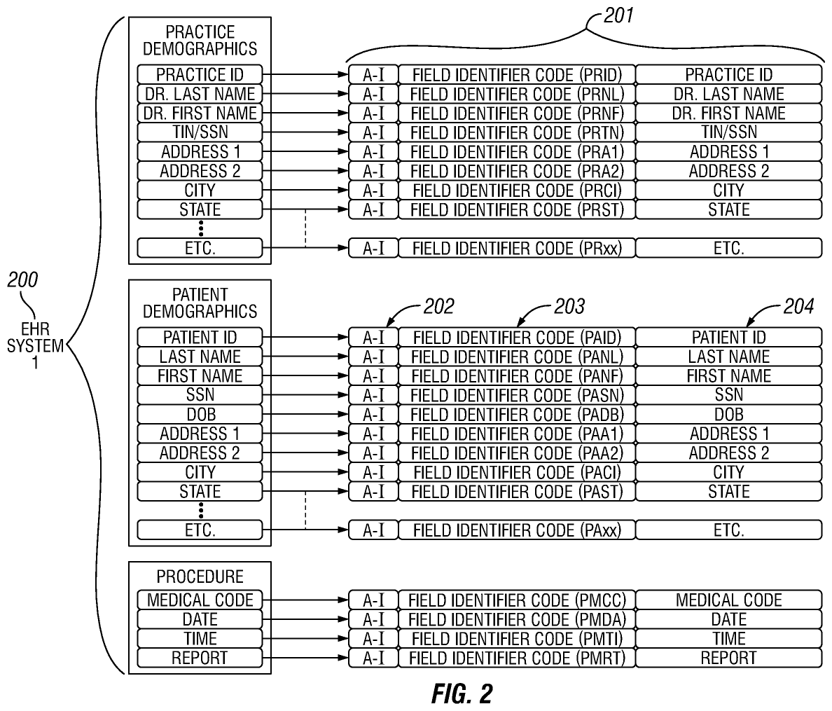

In addition to creating an A-I code 101, the Field Identifier Code (FIC) code will identify the source of the data for each field extracted from an EHR. This FIC indicates the source of the field data from the original EHR 100. FIG. 2 illustrates a diagram of individual data fields exported from an EHR in accordance with the disclosed principles to a first EHR system, where each individual data field contains an A-I code and a FIC. As illustrated, FIG. 2 shows an example of data files 201 extracted and created from an EHR 200 using the disclosed principles. Each extracted data field, in this illustrated embodiment, is composed of an A-I header 202 for the specific EHR/medical event, followed by the exemplary FIC code 203 corresponding to the source field of the extracted data 204 from that specific EHR. Other extracted data fields may also be included. Extracted field data files are stored external to the EHR system the data was extracted from. Each such extracted data field is thus used by the disclosed principles to create unique data files 201, with each having a uniquely created A-I tied to the specific medical event of the EHR, a FIC identifying the source field for the data in each data file 201, and the actual raw data from the source filed identified by the FIC. This results in the information within a distinct EHR being converted into unstructured data files 201 that provide the same information as the EHR that was created in a specific, structured data format that is not compatible with all EHR systems.

The data files can be stored on any type of storage device, including portable devices that can be used to share data between non-connected EHR systems, as well as other forms of local, centralized, distributed, or cloud-based storage and access systems. Storing the data on portable memory devices allows ready access to a patient's medical records, and can be carried or worn by a patient with a known medical condition, so as to allow medical history information to be readily available even in emergency and life-critical situations. Adding smart card features to the portable memory device allows easier access to centralized and cloud-based storage of data sets too large to be included on the portable memory device, such as MRIs, CATs, angiograms, etc. By adding smart card features to the portable memory device, centralized storage and cloud-based operation allows access to medical records by all affiliated providers, and system enhancements and bug fixes can be made available remotely and at the same time to all organizations that use the system.

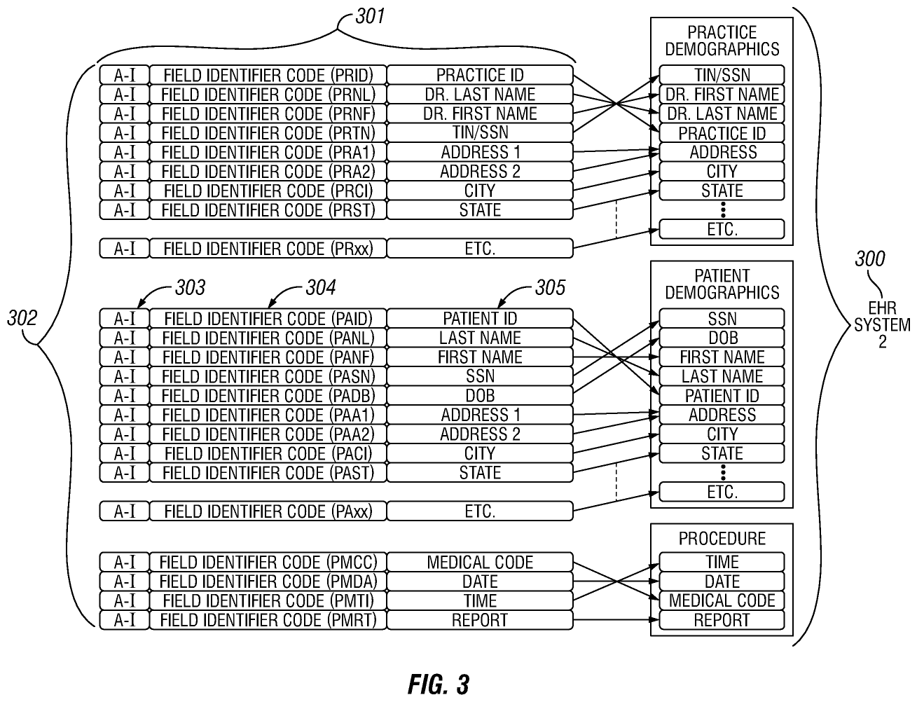

An EHR can be reconstructed from a set of data files stored as unstructured data in the SCUTWORK format. The A-I code is used to identify all files associated with a specific original EHR/medical event. The FIC identifies the original EHR source field for the data in the file. This configuration allows for reconstruction of an EHR from a set of data files regardless of the structure and order of fields used in a particular EHR system. FIG. 3 illustrates a diagram showing how a data field previously exported to a file from an EHR (such as the unstructured data files 201 created in FIG. 2) can be imported into a second EHR system, where fields identified as belonging to a specific EHR, as labeled by an A-I header, are further identified by a FIC that indicates the type of data contained within the file. The combination of the A-I code with the FIC allows a differently structured EHR system to correctly reconstruct a patient's EHR from the individual data files created and saved in accordance with the disclosed principles.

FIG. 3 details how such a reconstruction of an EHR 300 that is different from the original EHR 200 can be accomplished. FIG. 3 shows the files from the original EHR 200 in the same order (top to bottom) 302 as originally extracted. Each data field contains the A-I code 303 which identifies the specific original EHR 200, the FIC 304 which identifies the original EHR field, and each field data 305. The combination of the A-I 303 together with the FIC 304 in each data file 301 allows another EHR system 300 to import the data files and place each data field 305 into the correct field within the importing EHR system 300. For the purposes of illustration, FIG. 3 shows how data fields such as a provider's Practice ID, doctor name, etc., and data fields such a patient's social security number, date of birth, etc., can be organized/structured/formatted differently in a second EHR system 300 as compared to these same data fields found in the first EHR system 200 of FIG. 2. However, using the disclosed principles, this same data can be restructured from the unique data files 201, 301, created from the first EHR system 200 using these same disclosed principles, into the data organization/structure/format used by the second EHR system 300 without losing any data. Each data file 301 contains a specific piece of data 305 pertaining to a medical event memorialized in the specific EHR that was broken down. The A-I header 303 in each data file 301 identifies the patient's specific EHR to which the data 305 pertains, and the FIC 304 in each data file 301 identifies what field the raw data in each file 301 belongs to. Thus, the second EHR system 300 employs the SCUTWORK tool disclosed herein to rebuild the EHR into a format (i.e., structure) that is readable by the second system 300 using the unstructured data the SCUTWORK tool originally created from the EHR data of the first EHR system, regardless of the format that the first system employs.

Accordingly, the SCUTWORK tool defines and creates the A-I labels and the elemental data sets or files of the standard medical record. The uniquely A-I labeled elemental data sets can be cut from their EHR string data tree and stored randomly (unconnected and unstructured) on a patient-secured storage medium. The SCUTWORK tool is used to cut the string data trees of conventional EHRs to produce randomly saved A-I-labeled data sets on the storage device, and to reorganize randomly saved A-I labeled data sets from the patient's storage device into its string data tree to reconstruct the EHR. A different, subsequent SCUTWORK-capable EHR system can then identify the storage medium's A-I labeled data sets and place them into its string data tree. The disclosed principles permanently label every elemental data set of the EHR using such accountability information, which then allows the SCUTWORK-labeled data sets to be shared with any other SCUTWORK capable EHR system. Stated another way, a SCUTWORK-capable system can identify the A-I of each data set and reassemble the data sets into the different shaped data tree of a different EHR system.

The above process can be repeated between the importing EHR system 300 and additional EHR systems, each having their own unique format, since a new A-I code 101 would be generated when exporting from the most recent exporting EHR system. Any newly exported data can be returned to the original EHR system 200 for updating of records or shared with another, even incompatible, EHR system for additional medical related events. Additionally, existing EHR systems can be updated to both export and import medical records using the SCUTWORK format and technique by simply a software update. Once an EHR system is updated with SCUTWORK capabilities, there is no need to create conversion software to enable importing or exporting EHR records between two paired but incompatible EHR systems. In addition, SCUTWORK facilitates less expensive EHR systems so they can be utilized by small practices. Only systems that are not SCUTWORK-capable need to be modified. Existing systems can be updated. Relatively quick software development is all that is needed to add SCUTWORK functionality to existing EHR systems due to the simplicity of the concept.

Furthermore, the disclosed principles allow for the traditional technique of saving health records separately based on location; however, the disclosed principles provide for labeling all information produced with the identifying information regarding the professionals authoring the data. For example, SCUTWORK may label every part of the medical record with location, date, and the name of its author. Unique labeling of elemental data sets with such accountability information means the author responsible for the entry will be permanently recorded. All subsequent users of the information will have access to this information. This gives recognition of the entry of information to the author, who by allowing the information to be included in the SCUTWORK portable data storage device, gives permission for the patient to share this with other health providers, health plans, and health clearinghouses. This will improve the transparency and responsibility of the electronic health record.

Consequently, this allows all records to be combined into one “super record” of medical events of a patient, regardless of the provider, event location, or procedure type. Thus, extracting individual fields from an EHR and storing each of the individual fields as a separate file external to the EHR system is not limited to contents of a single HER or a single provider, etc. Patient data can be stored in the SCUTWORK format over a period of time, including a person's entire lifetime, to allow for storing a complete historical medical record. In this way, a provider allowed access by the patient can immediately see all services the patient has received his/her entire life, as well as when, by whom and where the service was performed. This allows all of the record to be discovered and for most, if not all, of the resources wasted by duplication of services to be eliminated.

Since each elemental medical procedure (such as, but not limited to, inoculations or blood pressure) are stored separately and labeled to identify the specific procedure, it is possible to scan through historical records to select these specific procedures in order to compile a comprehensive historical record. Thus, SCUTWORK-capable systems can select and import only the information needed from a group of SCUTWORK coded files, such as specific vaccination records. What information is included on the portable storage device will be the choice of the health care provider and patient. With SCUTWORK, the information from each of these electronic records could be kept separate or, as the provider chooses, they could be combined to make a super record.

For example, if the user wanted to see the patient's chronologic presentation of white blood cells, they could see a lifelong complete listing. A patient's entire life's vaccination history from all locations could be seen on one page using the deconstruction and reconstruction of data sets from EHRs provided by the disclosed principles. For example, searching for multiple occurrences of the same procedure, such as a pneumonia vaccine immunization over a period of years, would be relatively fast by searching an individual's SCUTWORK-created records for a particular procedure, such as a specific A-I followed by the Healthcare Common Procedure Coding System (HCPCS) HCPS code. The HCPS codes are a set of health care procedure codes based on the American Medical Association's Current Procedural Terminology (CPT). Individual medical services, procedures, equipment, etc. may be categorized and labeled by such a standard medical code within the SCUTWORK format. Using a standard medical coding system to identify specific types of SCUTWORK data files provides a fast and easy way to identify specific types of SCUTWORK health records.

Additionally, SCUTWORK-created unstructured data files may function with local, portable, or cloud storage of the SCUTWORK patient data records. SCUTWORK EHR data can be exported and stored on portable storage devices which can be carried by the patient. For the case of portable storage carried by the patient, SCUTWORK facilitates emergency access to the patient's data record at all times, since the patient can carry their own medical history with them, and there is no need to have access to an external database system to gain access to their medical records. For example, SCUTWORK-based patient portable storage devices can be created holding their medical records. The patient secured portable storage device allows the patient to choose what level of security they want for their health records. For example, for those who choose the same security as for their driver's license, credit cards, and social security number, then it could be kept without a password or code in their wallet for immediate access of the health record by health care workers in emergency circumstances. However, for those patients wishing high security then it could have a password and/or code limiting access to only those they consciously choose to share this information. For emergency situations, the patient-created security could be biometric, such as a fingerprint scan or a facial recognition scan, where the patient's portable records can still be accessed if the patient is unconscious or similarly incapacitated. The emergency personnel can use the device to immediately access the medical record and discover allergies, medications, and medical problems that could be potentially lifesaving.

Labeling data records in the SCUTWORK format is not necessarily limited to the field of healthcare or patient health records. The disclosed SCUTWORK systems and methods are an electronic record Rosetta Stone for any standardized record keeping profession. This includes the fields of health care, finance, law enforcement, engineering, accounting, banking, and so on. The SCUTWORK formatting and labelling of exported data sets/files can be applied to other fields, such as but not limited to, engineering, architecture, and construction. As such, SCUTWORK is designed for a standard record profession. Thus, SCUTWORK labeling can be applied to various fields that use similar standardized reporting where data interchange between incompatible systems is needed. Conventionally, when such standard record is made into an electronic record, all differently structured data electronic record versions will have similar record organization and formatting, but not similar structured electronic data organization. As SCUTWORK uses accountability information to label elemental data blocks, the structured data of each different electronic record can be reduced to unstructured data (without being a complete patient record) in accordance with the disclosed principles. This labeled unstructured data can then be reorganized back into a different electronic record having the exact same information as the EHR from which the unstructured data was derived.

Based on the above, exemplary aspects in accordance with the disclosed principles may comprise one or more of the following principles:

An interoperable healthcare data exchange method for enabling transferring of Electronic Health Records (EHRs) between incompatible EHR systems, the method comprising:
identifying each elemental part, and each data field within each elemental part, within a source EHR of a patient;
creating a unique Accountability Indicator (A-I) using multiple key EHR fields to uniquely identify the source EHR;
creating a string for each set in the EHR by concatenating the EHR A-I, a Field Indicator Code (FIC) to identify the data field for each set, and the data from each data field; and
storing each concatenated string as an individual data file, wherein each said data file can then be used to reassemble the source EHR whether in the originating EHR system or in a differently structured EHR from another system.

In some embodiments, each unique A-I code is a fixed length.

In some embodiments, each such FIC is a text string.

In some embodiments, each such FIC is a number.

In some embodiments, each concatenated string is saved on a portable device.

A method of reconstructing an EHR from individual files previously exported from an EHR system, the method comprising:
identifying unstructured data files associated with a particular EHR by locating files with a unique A-I header;
reading each identified data file and identifying the type of data stored within each data file by using a FIC included in each data file;
extracting the data stored within each data file; and storing the extracted data from the data file in a data field of an EHR, the proper field being identified by the FIC code contained within the data file.

In some embodiments, a new EHR is created and populated with the information contained within each file.

In some embodiments, an existing EHR is updated with the information contained within each file.

The foregoing description has made reference to several exemplary embodiments. It is understood, however, that the words that have been used are for description and illustration, rather than words of limitation. Thus, the present embodiments are therefore to be considered in all respects as illustrative and not restrictive. Changes may be made within the purview of the appended claims, as presently stated and as amended, without departing from the scope and spirit of the disclosure in all its aspects. All changes which come within the meaning and range of equivalency of the claims are therefore intended to be embraced therein. Although this description makes reference to particular means, materials and embodiments, the disclosure is not intended to be limited to the particulars disclosed; rather, the disclosure extends to all functionally equivalent technologies, structures, methods and uses such as are within the scope of the appended claims. Further, the recitation of any method steps does not denote a particular sequence for execution of the steps. Such method steps may therefore be performed in a sequence other than recited unless the particular claim expressly states otherwise.

In the numerous embodiments of the inventive subject matter disclosed herein, such embodiments may be referred to herein, individually and/or collectively, by the term “invention” merely for convenience and without intending to voluntarily limit the scope of this application to any single invention or inventive concept if more than one is in fact disclosed. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

Moreover, the Abstract is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the claims reflect, inventive subject matter lies in less than all features of a single disclosed embodiment. Thus, the following claims are hereby incorporated into the Detailed Description, with each claim standing on its own as a separate embodiment.

1. A method of creating unstructured data files comprising information in Electronic Health Records (EHRs), the method comprising:
identifying a source EHR of a patient, wherein the source EHR comprises one or more elemental parts, each elemental part comprising a plurality of data fields where each data field has data stored therein;
creating a unique Accountability Indicator (A-I) using data stored within certain ones of the plurality of data fields that uniquely identify the source EHR; and
creating a data file for each data field of the source EHR, each data file comprising the unique A-I, a Field Indicator Code (FIC) identifying each said data field, and the data from each said data field.

2. A method in accordance with claim 1, wherein the elemental parts each comprise patient identifying information, provider identifying information, or information identifying a medical procedure or event.

3. A method in accordance with claim 1, wherein the data fields are selected from the group consisting of patient name, patient address, patient social security number, patient date of birth, date of a medical procedure or event, medical code of a medical procedure or event, date of a medical procedure or event, report associated with a medical procedure or event, provider name, provider identification number, or provider address.

4. A method in accordance with claim 1, wherein the certain ones of the plurality of data fields that uniquely identify the source EHR comprise at least patient name, patient social security number, patient date of birth, type of medical procedure or event, date of medical procedure or event, and provider name.

5. A method in accordance with claim 1, wherein creating a data file comprises creating a concatenated data string for each data field of the source EHR.

6. A method in accordance with claim 1, wherein the A-I comprises a header for each data string created for each data file.

7. A method in accordance with claim 6, wherein creating a unique A-I for each data string further comprises creating a unique A-I of a fixed length for each data string.

8. A method in accordance with claim 1, wherein each FIC comprises a text string.

9. A method in accordance with claim 1, wherein each FIC comprises a number.

10. A method in accordance with claim 1, further comprising storing each created data file on a portable storage device.

11. A method in accordance with claim 10, further comprising securing said portable storage device using biometric information of the patient.

12. A method in accordance with claim 1, further comprising storing a cloud-based record associated with a data field of a created data file.

13. A method of reconstructing a patient EHR previously deconstructed into unstructured data files, the method comprising:
identifying ones of the unstructured data files having a single unique Accountability Indicator (A-I) derived from certain ones of a plurality of data fields identifying the deconstructed EHR;
identifying Field Identifier Codes (FICs) within the identified ones of the unstructured data files, along with stored data corresponding to each identified FIC, the FICs corresponding to data fields of a deconstructed EHR;
extracting the data stored with each identified FIC; and
storing the extracted data in corresponding data fields of a reconstructed EHR identified by each identified FIC.

14. A method in accordance with claim 13, wherein the data fields are selected from the group consisting of patient name, patient address, patient social security number, patient date of birth, date of a medical procedure or event, medical code of a medical procedure or event, date of a medical procedure or event, report associated with a medical procedure or event, provider name, provider identification number, or provider address.

15. A method in accordance with claim 13, wherein the certain ones of the plurality of data fields that uniquely identify the deconstructed EHR comprise at least patient name, patient social security number, patient date of birth, type of medical procedure or event, date of medical procedure or event, and provider name.

16. A method in accordance with claim 13, wherein each data file comprises a concatenated data string for each data field of the deconstructed EHR.

17. A method in accordance with claim 16, wherein the identified unique A-I comprises a header for each data string.

18. A method in accordance with claim 13, wherein the data files are read from a portable storage device.

19. A method in accordance with claim 18, further comprising accessing said portable storage device using biometric information of the patient.

20. A method in accordance with claim 19, further comprising accessing a cloud-based record associated with the reconstructed EHR after reconstructing the EHR from the portable storage device.
